(19) United States
(12) Patent Application Publication
(10) Pub. No.: US 2019/0275236 A1
(43) Pub. Date: Sep. 12, 2019

(54) FULL PATH RIGID NEEDLE

(71) Applicants: Eyal Barmaimon, Haifa (IL); Lior Shtram, Tel-Aviv (IL); Shai Finkman, Haifa (IL); Nadav Cohen, Haifa (IL); Leon Slobitker, Carmiel (IL)

(72) Inventors: Eyal Barmaimon, Haifa (IL); Lior Shtram, Tel-Aviv (IL); Shai Finkman, Haifa (IL); Nadav Cohen, Haifa (IL); Leon Slobitker, Carmiel (IL)

(21) Appl. No.: 16/271,359

(22) Filed: Feb. 8, 2019

Related U.S. Application Data
(60) Provisional application No. 62/640,423, filed on Mar. 8, 2018.

(51) Int. Cl. A61M 5/158; A61M 5/14; A61M 5/162

(52) U.S. Cl. CPC A61M 5/158; A61M 5/1413; A61M 2005/1581; A61M 2005/1585; A61M 5/162

(57) ABSTRACT

An apparatus and method for the aseptic delivery of a solution in a wearable form factor is disclosed. A patch pump may include a start button that, upon activation, causes an elastic fluid path to move. The elastic fluid path may include a first end that moves along a first plane to penetrate a solution container as well as a second end that moves, concurrently, along a second plane to penetrate a user's tissue. The elastic fluid path may maintain its flow resistance during the concurrent movement. The patch pump's housing may include a door with an opening configured to enable the second end to penetrate a user via the opening. The door may open when the device is removed from the user such that a portion of the open door may extend further than the second end of the elastic fluid path to prevent exposure of the second end.

This application claims the benefit of U.S. Provisional Application No. 62/640,423 having a filing date of Mar. 8, 2018, which is incorporated by reference as if fully set forth.

### BACKGROUND

There is a strong market need for an apparatus that can enable the subcutaneous self-administration of solutions such as medication in a wearable format factor. For instance, the treatment of diabetes requires the subcutaneous delivery of insulin. As a result, wearable pumps that deliver a medication to a patient may be used to administer such solutions. These pumps may incorporate the medication, pumping mechanism, and infusion set into a patch that attaches to a patient's skin, thus eliminating the need for external systems.

Patients may wear patch pumps for a prolonged period of time and, accordingly, patient comfort and ease of use is a consideration in the design and manufacturing of such patch pumps.

### SUMMARY

An aspect of the disclosed subject matter relates to an apparatus and method for the delivery of a solution in a wearable form factor such as a patch pump. A patch pump may include a start button that, upon activation, causes an elastic fluid path to move. The elastic fluid path may include a first end that moves along a first plane to penetrate a solution container as well as a second end that moves, concurrently, along a second plane to penetrate a user's tissue. The elastic fluid path may maintain its flow resistance during the concurrent movement. The patch pump's housing may include a door with an opening configured to enable the second end to penetrate a user via the opening. The door may open when the device is removed from the user such that a portion of the open door may extend further than the second end of the elastic fluid path to prevent exposure of the second end.

### BRIEF DESCRIPTION OF THE DRAWINGS

A more detailed understanding may be had from the following description, given by way of example in conjunction with the accompanying drawings wherein:

FIG. 1A is a graphic depiction of a patch pump on a user.

FIG. 1B is a graphic depiction of the outer casing of an embodiment of a patch pump.

FIG. 1C is a graphic depiction of an embodiment of a patch pump with the outer casing removed.

FIG. 1D is a graphic depiction of an embodiment of a patch pump with a sterilized and non-sterilized portion.

FIG. 2A is a graphical depiction of a patch pump including an elastic fluid path in a first position.

FIG. 2B is a graphical depiction of a patch pump including an elastic fluid path in a second position.

FIG. 2C is a cross sectional view of a patch pump including an elastic fluid path.

FIG. 3A is a cross sectional view of a patch pump including a door with a release.

FIG. 3B is a bottom view of a patch pump including a door and an opening.

FIG. 3C is a cross sectional view of a patch pump including a released door.

FIG. 4 is a flow chart for use of a patch pump including a door.

### DETAILED DESCRIPTION

Examples of different pumps and needle insertion mechanism implementations will be described more fully hereinafter with reference to the accompanying drawings. These examples are not mutually exclusive, and features found in one example can be combined with features found in one or more other examples to achieve additional implementations. Accordingly, it will be understood that the examples shown in the accompanying drawings are provided for illustrative purposes only and they are not intended to limit the disclosure in any way. Like numbers refer to like elements throughout.

Below are described an apparatus and methods for delivering solutions such as pharmaceutical drugs and/or biologics to a patient. The apparatus and methods may allow for prescription or standard off-the-shelf drug cartridges to be utilized without compromising the sterility of the solution. This may enable a patient to self-administer a solution in a self-contained wearable patch pump form factor that is cost effective and comfortable to wear. The disclosure subject matter provided herein may allow the manufacturing and use of a patch pump that is small enough in size to be comfortable to wear.

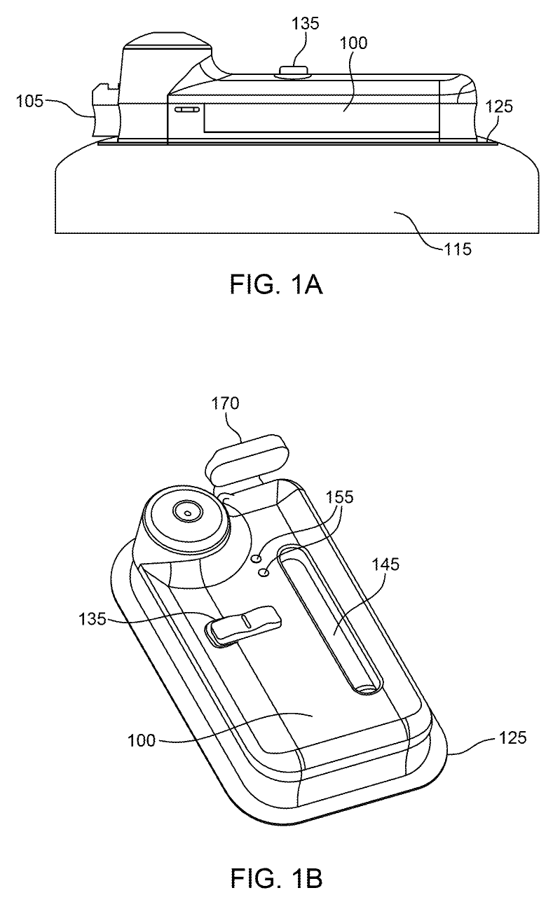

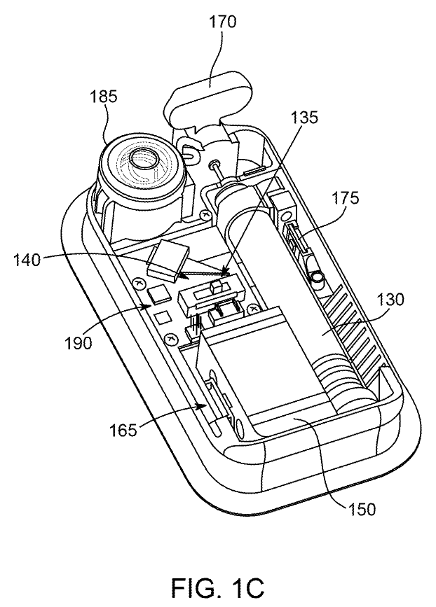

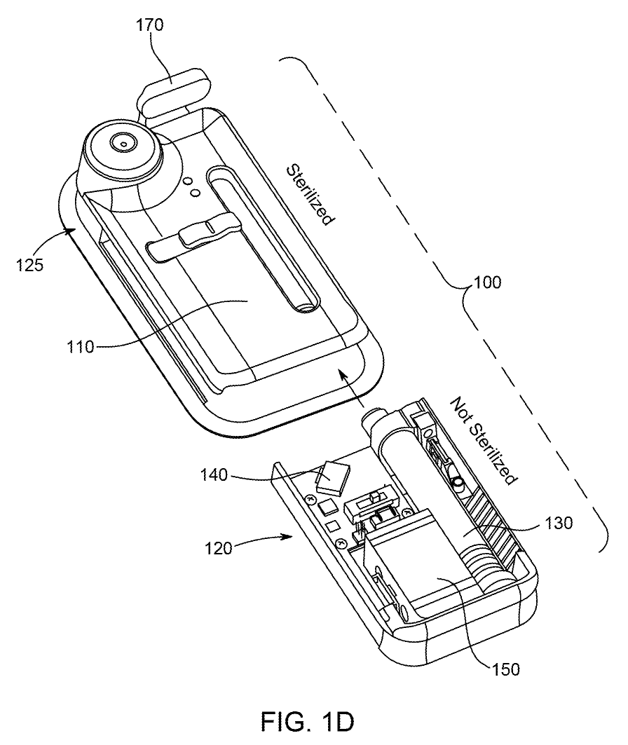

FIG. 1A is a graphic depiction of a patch pump 100 on a patient. 115. The patch pump 100 includes a base 125 that contacts the patient's skin. In some embodiments, the base 125 includes an adhesive liner that affixes the patch pump 100 to the patient 115. The patch pump 100 may further include a user flow control switch 135. The user flow control switch 135 may enable a user to pause and control the flow rate of a solution. The patch pump 100 further includes a start button 105 that may be pressed by a user to cause a cannula and/or needle to be inserted into the patient and the solution to flow from a drug container 130 through the cannula and/or needle into the patient.

As shown in FIG. 1B, the patch pump 100 may further include a drug viewing window 145. This viewing window may enable a user to view the amount of a solution that remains in the drug container 130. The patch pump 100 may also include one or more visual indicators 155. The visual indicators 155 provide feedback on the operational status of the system. The operational status of the system may include warnings such as an over/under temperature warning, drug expiration warning and over/under pressure warning. In addition, the operational status may include information indicating that the drug is being administered, how much time is remaining for the drug dosage to be completed and the current flow rate. The visual indicators may include LEDS, LCD displays or other similar display technologies known in the art. The information that is displayed by visual indicators may also be wirelessly transmitted to a mobile computing device such as a smart phone utilizing any of the wireless communication methods known in the art.

The patch pump 100 may further include a removable safety 170. The removable safety mechanically engages the start button 105 and prohibits the start button 105 from being involuntary pressed.

FIG. 1C shows additional components of a patch pump 100. The patch pump 100 may also include a needle insertion component 185 that is mechanically connected to the start button 105. In addition, the patch pump 100 includes an electronic circuit board 140 that includes control circuitry for the visual indicators 155, user flow control switch 135 and a pressurization system electro mechanical unit 150. In addition, the electronic circuit board 140 may be communicatively connected to one or more sensors. These sensors may include a proximity sensor 175. The electronic circuit board 140 may also include a memory 190. The memory 190 may store dosing instructions for the administration of the solution. In addition, the memory 190 may also store information regarding the administration of the solution. This information may include, time, date, and flow rate when the solution was administered. The electronic circuit board 140 may control the visual indicators 155 and electro mechanical unit 150 based on the information stored in the memory and the feedback from the sensors. The electronic circuit board 140 may include a communication module that enables the transmission of information stored in the memory 190 to a wireless computing device. In addition, the communication module may also receive updated dosing instructions that are subsequently stored in the memory 190.

As shown in FIG. 1D, the patch pump 100 may include a sterilized assembly 110 and a non-sterilized assembly 120. The sterilized assembly 110 may include the needle insertion component 185 the start button 105, the removable safety 170 and the base 125. The sterilized assembly 110 may be sterilized by gamma radiation or any other similar technique. The non-sterilized assembly 120 includes the electro mechanical unit 150, electronic circuit board 140 and the drug container 130. The drug container 130 contains a solution that was filled under aseptic conditions. Therefore, the inner part of the drug container 130 that contains solution is sterilized but the outer part of the drug container 130 is not. This enables the drug container 130 to be handled under non-aseptic conditions. The non-sterilized assembly 120 is configured to mechanically couple to and fit inside of the sterilized assembly 110.

A device, such as a patch pump, configured in accordance with the disclosed subject matter may consume a smaller footprint than traditional patch pumps and/or fluid delivery mechanism. Such a device may be configured to allow an elastic fluid path's first end, which comprises a rigid needle, to move in a first plane and the elastic fluid path's second end, which also comprises a rigid needle, to move in a second plane. The first end may penetrate a solution container based on the movement in the first direction and the second end may penetrate a user's tissue based on the movement in the second direction. The movement may be caused by the activation of a start button such as, for example, via a push of the start button. As an example, a patch pump start button may be pushed and the push may cause the first end of an elastic fluid path to penetrate the lining of a drug container. The push may also cause the second end of an elastic fluid path to be released and to penetrate the tissue of a user. Accordingly, a fluid path may be created from the solution container to a user.

Additionally, the device may include a door on the bottom of the device such that the door may be configured to remain closed until the start button is activated. Upon activating the start button, a door release may be engaged and enable the door to open when the door is not experiencing a pressure against it, such as when the device is removed from the body of a user. The door may contain an opening which allows the second end of the elastic fluid path to traverse through the door and penetrate the tissue of a user while the device is in contact with the user. If the contact between the user and the device is broken, the door may slide open such that the door extends past the second end of the elastic fluid path and prevents the second end of the elastic fluid path from being exposed and mitigating or preventing accidental contact with the second end of the elastic fluid path.

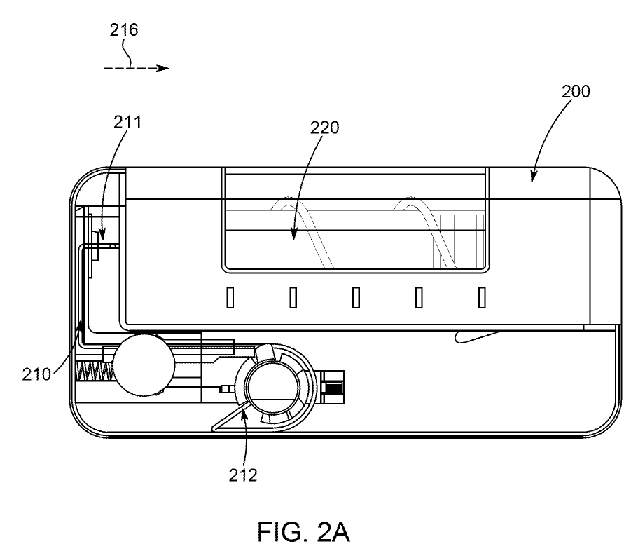

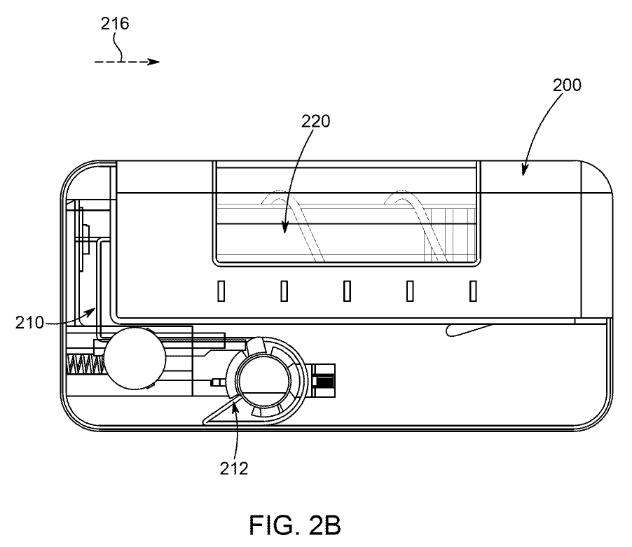

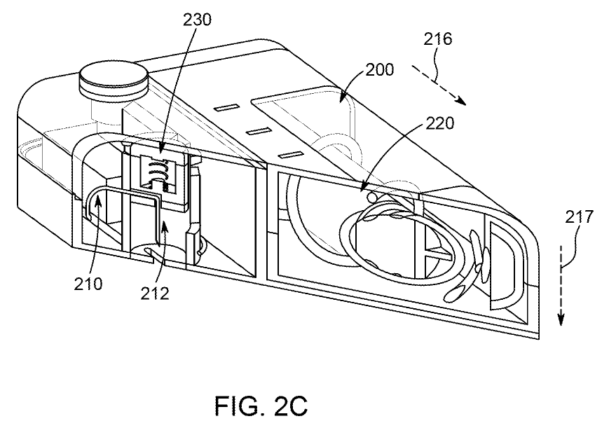

FIG. 2A shows a diagram of a patch pump 200 that includes an elastic fluid path 210. The patch pump 200 may be the same as or similar to patch pump 100 of FIGS. 1A-D or may be different from and include components different from patch pump 100. The elastic fluid path 210 may be included in the needle insertion component 185, as shown in FIGS. 1A-D or may be part of a different patch pump or other solution delivery mechanism which requires the use of an elastic fluid path 210. Accordingly, the elastic fluid path 210 may be configured to operate with the components shown in FIG. 1A-D or, alternatively, may be configured to operate with a subset of the components shown in FIG. 1A-D or none of the components shown in FIG. 1A-D.

FIG. 2A shows a patch pump 200 and an elastic fluid path 210. The fluid path 210 contains a first end 211 and a second end 212. The first end 211 and second end 212 may comprise one or more rigid needles such that the rigid needles are configured to puncture one or more surfaces. The elastic fluid path 210 may be made of the same material or may be made of a combination of materials.

The elastic fluid path 210 may be configured such that the first end 211 and second end 212 can move in two different planes, concurrently. The first end 211 may be configured to move in a first plane in a direction such that the first end 211 comes into contact with and punctures a fluid container 220. The second end 212 may be configured to move in a second plane in a direction such that the second end 212 punctures the tissue of a user

As an example, as shown in FIGS. 2A and 2B, the first end 211 of the elastic fluid path 210 may move in a horizontal direction 216 to puncture a membrane of the fluid container 220. The first end 211 may start at a first position, as shown in FIG. 2A and move towards the right in direction 216 to a second position, as show in 2B, where the first end 211 (not shown in FIG. 2B) punctures a membrane of the fluid container 220. The second end 212 of the elastic fluid path 210 may move in a vertical direction to puncture a tissue of a user (not shown). In reference to FIG. 2A, the second end 212 may start at a first position and move back towards the bottom of the patch pump 200 to a second position.

The movement of the elastic fluid path 210 may be caused by the activation of a start button, such as the start button 105 from FIG. 1A. The start button may exert a force, caused by a mechanical or electronic movement, against the elastic fluid path 210 that causes the first end 211 to puncture the fluid container 220. Additionally, the activation of the start button may cause a needle release mechanism 230 to exert a force against the second end 212 of the elastic fluid path 210 to cause the second end 212 to move towards the bottom of the push pump 200 and puncture a user's tissue.

FIG. 2C shows a cross sectional view of the patch pump 200. As shown, the second end 212 of an elastic fluid path 210 may traverse a path indicated by the arrow 217. The first end 211 (not shown) of the elastic fluid path 210 traverses a path indicated by the arrow 216. Notably, the first end 211 and second end 212 of the elastic fluid path 210 may move concurrently, in two different planes, as disclosed herein.

As shown in FIGS. 2A-C, the first end 211 and second end 212 may move along different planes. The planes may be orthogonal to each other such as, for example, as shown in FIGS. 2A-C where the first end 211 moves along a plane in the direction indicated by the arrow 216 and the second end 212 moves along a plane in the direction indicated by arrow 217. As shown, the plane corresponding to the first end 211 is a 90 degrees change in direction from the plane corresponding to the second end 212. A patch pump 200 that includes an elastic fluid path 210 with a 90-degree change in direction may occupy a smaller footprint in a first dimension than an elastic fluid path 210 with a smaller (e.g., 60 degree) change in direction. For example, a patch pump 210 with an elastic fluid path 210 with a 90-degree change in direction may be shorter in length than a similar needle patch pump 200 with an elastic fluid path 210 with a 60-degree change in direction. However, a patch pump 200 that includes an elastic fluid path 210 with a 90-degree change in direction may occupy a larger footprint in a second dimension than an elastic fluid path 210 with a smaller (e.g., 60 degree) change in direction. Continuing the example, the patch pump 200 with an elastic fluid path 210 with a 90-degree change in direction may be lower in height than a similar patch pump 200 with an elastic fluid path 210 with a 60-degree change in direction.

The elastic fluid path 210 may maintain its fluid flow resistance during and/or after movement of at least the first end 211 and second end 212. The fluid flow resistance may be maintained as a result of the path in the elastic fluid path 210 maintaining its structural and/or special integrity, such as its structural shape, while the first end 211 and second end 212 move. Maintaining the fluid flow resistance during and/or after the movement may allow the solution to enter a user's body in at an intended rate without slowing down the flow or causing breaks in the flow.

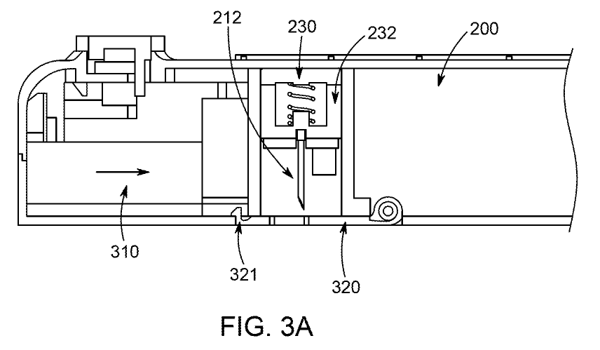

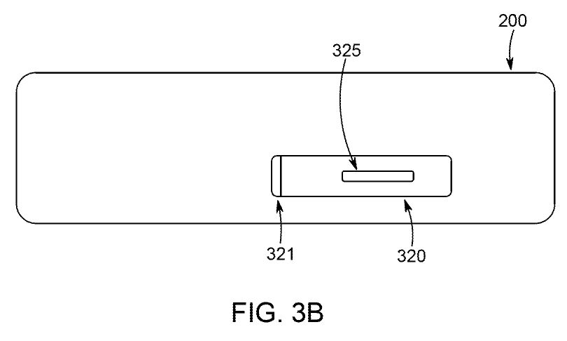

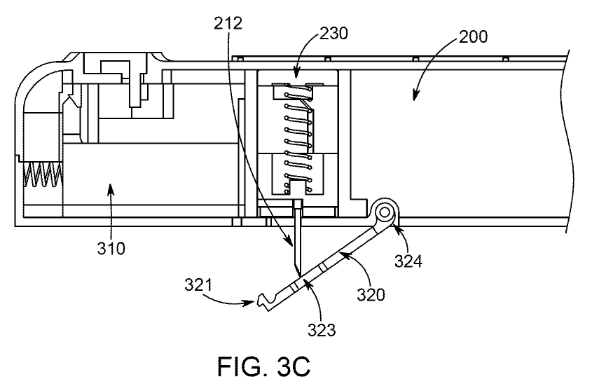

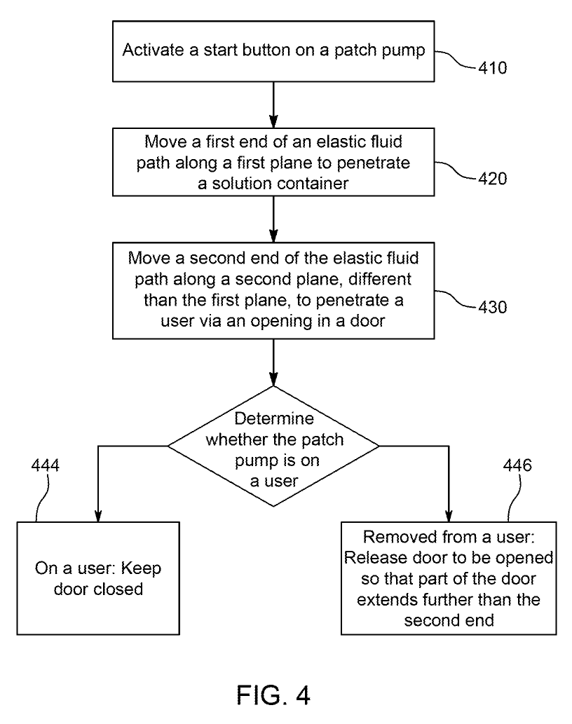

FIG. 3A shows the patch pump 200 of FIGS. 2A-C and the second end 212 of the elastic fluid path 210 (210 not shown in FIGS. 3A-C). As shown in FIG. 3A, the patch pump 220 may include a door 320 positioned below the second end 212 of the elastic fluid path 210. The door 320 may be configured to remain in a closed position, as shown in FIG. 3A and may be held in the closed position as a result of a release 321 which prevents the door from opening.

A movable component 310 of the patch pump 200 may be located in proximity to the release 321. The movable component 310 may move towards the release 321 upon activation of a start button (not shown). For example, the force used to push the start button to activate it may cause the movable component 310 to move from a first position to a second position. During the movement from a first position shown in FIG. 3A to a second position shown in FIG. 3C, the movable component 310 may come in contact with the release 321 such that it causes the release to activate. An activated release may no longer prevent the door 320 from opening. It should be noted, though, that an activated release may not cause a door to open but, rather, simply no longer prevent the door from opening, as further discussed herein. FIG. 3C shows the movable component 310 in a second position after movement caused by activation of the start button. As shown, the release 321 may be activated such that the door 320 is able to open.

The door 320, as shown in FIG. 3A may be positioned on the bottom of the patch pump 200 such that it is placed on or in close proximity to a user. Specifically, the door may be in contact or in close proximity to the tissue of a user through which the second end 212 of the elastic fluid path 210 penetrates as a result of its movement.

As shown in FIG. 3B the door 320 may include an opening 325. The opening 325 may be wide enough to allow the second end 212 of the elastic fluid path 210 to traverse through the door 320 and to penetrate a user's tissue.

The door 320 may remain in a closed position even after the release 321 has been activated as a result of a force applied on the door 320. The force applied on the door 320 may be provided by the surface, such as a user, on which the door 320 is placed. As shown in FIG. 3C, the door 320 may open. The door 320 may open when the release 321 has been activated and when the patch pump 200, and specifically the door 320, is removed from a surface, such as a user, that the door was placed against. The removal of the force provided by the surface, such as by a user, may enable the door to open. Additionally, according to an implementation, the door may be spring loaded via spring 324 such that the spring provides a force that causes the door 320 to open when the release 320 is activated and when a force, causing the door to remain closed, is removed.

According to an implementation, the door 320 may open such that the door 320 prevents the second end 312 of the fluid path 210 from being exposed to the environment. As shown, the door 320 may be configured to open up to a certain angle. The angle may be an angle that enables the door 320 to open such that the second end 212 of the elastic fluid path 210 rests on a portion of the door 320 such that the door extends past the second end 212. The portion of the door 320 may be any part of the door where the second end 212 of the elastic fluid path 210 is able to be in connection with and rest on. Alternatively, the door may include a needle rest 323 that is configured to connect with the second end 212 of the elastic fluid path. The door may be configured such that the door extends out further than the second end 212 of the elastic fluid path regardless of whether the second end 212 of the elastic fluid path 210 rests on any applicable portion of the door 320 or a specific needle rest portion 323.

The door 320 and/or the spring 324 may include a doorstopper which may be a physical component that blocks the door from opening such as, for example, a bump, a spring, or any other applicable stopper. The doorstopper may be configured to prevent the door 320 from extending past a given angle. The angle may be determined such that the angle enables the door to extend out further than the second end 212 of the elastic fluid path, as disclosed herein. Accordingly, the doorstopper may be configured based on the length of the second end 212 that protrudes past a plane representing the edge of the patch pump 200.

Notably, the configurations disclosed herein may prevent the second end 212 of the elastic fluid path 210 from protruding openly and being exposed when the patch pump 200 is removed from the body of a user or from any other surface. Preventing the second end 212 of the elastic fluid path 210 from protruding openly and being exposed may prevent unintended contact with the second end 212 and may prevent the second end 212 from puncturing unintended items.

Alternatively or in addition, the patch pump 200 may include a needle release mechanism 230, as shown in FIGS. 3A and 3C. The needle release mechanism 230 may be configured to propel the second end 212 of the elastic fluid path 210 in a downward direction towards the door 320. The needle release mechanism 230 may be configured to release the second end 212 based on the activation of a start button, such as the start button 170 of FIGS. 1A-B. Alternatively, the needle release mechanism 230 may be configured to release the second end 212 based on a different trigger or force. The trigger may be an electronic signal or may be a mechanical change or force.

The needle release mechanism 230 may include a needle blocker 232 configured to prevent the needle release mechanism 230 from releasing the second end 212 of the fluid path 210. The needle blocker 232 may prevent the release if the patch pump 200 is not placed on a user. A determination that the patch pump 200 is not placed on a user may be made based on a detection of the force applied against the patch pump 200, based on electronic, electrochemical, or mechanical signals that indicate that the patch pump 200 is not placed against a user, or by any other applicable technique. Alternatively, the needle blocker 232 may prevent the release if the door 320 is opened. A determination may be made that the door 320 is open based on applicable electronic signals or, alternatively, via mechanical components within the patch pump 200 and/or specifically the door 230, needle release mechanism 230, and needle blocker 232. By preventing the second end 212 of the fluid path 210 from being released when the patch pump 200 is not placed against a user or when the door 320 is opened may prevent the second end 212 from being exposed to the environment unintentionally, may prevent unintended contact with the second end 212 and may prevent the second end 212 from puncturing unintended items.

Although features and elements are described above in particular combinations, one of ordinary skill in the art will appreciate that each feature or element can be used alone or in any combination with the other features and elements.

What is claimed is:

1. A device comprising:
a start button;
an elastic fluid path structure comprising a first end and a second end configured to move concurrently wherein the first end is configured to move along a first plane to penetrate a solution container and the second end is configured to move along a second plane, different than the first plane, to penetrate a user, the elastic fluid path structure configured to maintain its flow resistance during the concurrent movement;
a housing comprising a door; and
the door comprising an opening configured to enable the second end to penetrate the user via the opening; and
wherein the door is configured to open when the device is removed from the user such that a portion of the open door extends further than the moved second end to prevent exposure of the second end.

2. The device of claim 1 wherein the first and second ends are rigid needles.

3. The device of claim 1, further comprising a door release configured to activate when the start button is activated wherein the door release enables the door to open when the device is removed from the user.

4. The device of claim 1, wherein the first plane is orthogonal to the second plane.

5. The device of claim 1, wherein the first plane is at an angel of between 60 and 90 degrees to the second plane.

6. The device of claim 1, wherein the first plane is at an angel of between 30 and 60 degrees to the second plane.

7. The device of claim 1, wherein the door is spring loaded.

8. The device of claim 1, wherein the door comprises a door stopper configured to prevent the door from opening past an extension point.

9. The device of claim 1, further comprising a needle blocker configured to prevent the second end of the elastic fluid path from moving along the second plane to penetrate a user if the device is removed from the user.

10. The device of claim 1, wherein the fluid flow resistance of the fluid path is maintained due to the structure of the fluid path maintain its structural shape.

11. The device of claim 1, wherein the door comprises a needle rest.

12. A method comprising:
activating a start button on a patch pump;
moving a first end of an elastic fluid path along a first plane to penetrate a solution container;
moving a second end of the elastic fluid path along a second plane, different than the first plane, to penetrate a user via an opening in a door;
maintaining a fluid path flow resistance while moving the first end and the second end;
on a condition that the patch pump is removed form a user, releasing the door to be opened such that a portion of the open door extends further than the moved second end to prevent exposure of the second end.

13. The method of claim 12 wherein the first and second ends are rigid needles.

14. The method of claim 12, further comprising activating a door release when the start button is activated wherein the door release enables the door to open when the device is removed from the user.

15. The method of claim 12, wherein the first plane is orthogonal to the second plane.

16. The method of claim 12, wherein the first plane is at an angel of between 60 and 90 degrees to the second plane.

17. The method of claim 12, wherein the first plane is at an angel of between 30 and 60 degrees to the second plane.

18. The method of claim 12, wherein the door is spring loaded.

19. The method of claim 12, wherein the fluid flow resistance of the fluid path is maintained due to the structure of the fluid path maintain its structural shape.

20. The method of claim 12, wherein the door comprises a needle rest.
